(19) United States
(12) Patent Application Publication
(10) Pub. No.: US 2021/0072706 A1
(43) Pub. Date: Mar. 11, 2021

(54) TIME MEASUREMENT APPARATUS, TIME MEASUREMENT METHOD AND METHOD FOR PRODUCING A TIME MEASUREMENT APPARATUS

(71) Applicant: T-TIMR GmbH, Hamburg (DE)

(72) Inventors: Christian Lieb, Ebensfeld (DE); Maximilan Engel, Wunsiedel (DE)

(21) Appl. No.: 17/040,576

(22) PCT Filed: Mar. 29, 2019
(86) PCT No.: PCT/EP2019/058063

§ 371 Date: Sep. 23, 2020

(30) Foreign Application Priority Data
Mar. 29, 2018 (DE) 10 2018 107 694.0

(51) Int. Cl. G04F 13/06; G01N 33/14; A47J 31/52

(52) U.S. Cl. CPC G04F 13/06; A47J 31/52; G01N 33/14

(57) ABSTRACT

Provided is a time measurement apparatus having a transport device for a liquid, at least one sensor and at least one display which is at least communicatively connected to the sensor, is optically freely accessible and is intended to display a contact duration of the time measurement apparatus with the liquid or an item of meta information derived from the contact duration. The transport device includes at least one reservoir region for receiving liquid from an environment of the time measurement apparatus through an entrance area, at least one detection region for detecting the liquid and at least one time region having a number of channels for transporting the liquid from the reservoir region to the detection region in a defined transport duration. The sensor is designed to detect the liquid in the detection region at an end of the time region which is remote from the reservoir region.

### CROSS-REFERENCE TO RELATED APPLICATIONS

This application claims priority to PCT Application No. PCT/EP2019/058063, having a filing date of Mar. 29, 2019, based on German Application No. 10 2018 107 694.0, having a filing date of Mar. 29, 2018, the entire contents both of which are hereby incorporated by reference.

### FIELD OF TECHNOLOGY

The following relates to a time measurement apparatus having a transport means, transport device or transporter for a liquid, at least one sensor and at least one display means or display which is at least communicatively connected to the sensor and is intended to display a contact duration of the time measurement apparatus with the liquid or an item of meta information derived from the contact duration. The transport means comprises at least one reservoir region for receiving liquid from an environment of the time measurement apparatus through an entrance area, at least one detection region for detecting the liquid and at least one time region having a number of channels for transporting the liquid from the reservoir region to the detection region in a defined transport duration. The sensor is designed to detect the liquid in the detection region at an end of the time region which is remote from the reservoir region.

The following relates also to a time measurement method with the time measurement apparatus and method for producing the time measurement apparatus.

### BACKGROUND

Preparing tea is crucially dependent on the time in which the freshly brewed tea can soak before the tea leaves or other plant components are removed from the water. Usually, this time has been measured hitherto with a clock, in particular with a timer. On one hand, the disadvantage with this is that a clock is not always available while preparing tea. On the other hand, the user has to know the ideal time that the selected tea needs to steep. Furthermore, this causes a problem, in that the time measured by the clock is not necessarily synchronised with the steeping time of the tea, for example because the clock was started only after a certain time has elapsed since infusing the tea.

The utility model DE202014009078U1 describes a tea preparation device for bag-based, single portion preparation of a tea drink, wherein the tea is routed through an extension of the teabag and a layer of food coloring into a discolorable non-woven fabric. With this, the non-woven fabric becomes increasingly discolored by the teabag with increasing time, so that a steeping time can be derived from this. A disadvantage of the tea preparation device is that the tea enters through the teabag in a relatively uncontrolled manner and is transferred with several changes of direction, which results in a poorly reproducible transport time of the tea to the non-woven fabric.

The disclosure document DE10151356A1 describes an apparatus and a method for measuring time, in particular for tea preparation. Again, in this case the tea is drawn through the teabag into a transport means, for example blotting paper. In the blotting paper, the tea is fed via several twisted paths on to colored markings on the rear of the blotting paper, which become visible through saturation of the blotting paper. Again, this causes disadvantages, in that the tea enters through the teabag in a relatively uncontrolled manner and is transferred with several changes of direction, which results in a poorly reproducible transport time of the tea to the colored markings.

Patent application DE102015108921A1 discloses a time measurement apparatus with a strip of absorbent material, which is coated with an airtight material, wherein the coating has a hole in the lower half, and wherein a dye is applied on the strip. If the strip is placed in tea, then the tea is sucked by the strip through the hole and the tea migrates into the strip through capillary forces. At the same time, it either takes with it on its path, for example, a dye applied in the vicinity of the hole or it changes the color of a pH indicator dye applied in advance at a given point. In any case, the user can understand the rise of the tea and hence the steeping time with the aid of the dye. However, this has the disadvantage that the tea does not necessarily penetrate the strip evenly and in a predictable amount, so that the time measurement is imprecise.

### Technical Problem

A difficulty is to create an economical and simply-built time measurement apparatus and a simple time measurement method for precise and reliable measurement of a steeping time for a tea. Another difficulty is also to create an economical and reliable method for producing the time measurement apparatus.

### Technical Solution

The following provides a time measurement apparatus, which solves the technical problem in accordance with the embodiments of the invention. In addition, the problem is solved by a time measurement method in accordance with the embodiments and a production method in accordance with the embodiments. Advantageous embodiments are subject of the dependent claims.

### Description of the Embodiments

The time measurement apparatus according to embodiments of the invention comprises a transport means for a liquid, at least one sensor and at least one display means connected at least communicatively with the sensor and optically freely accessible, to display a period during which the time measurement apparatus is in contact with the liquid or an item of meta information derived from the contact duration. If the time measurement apparatus, for example, is brought into contact with hot water simultaneously with a teabag for preparing tea, the steeping time for the tea equals the contact duration. If a user reads the contact duration from the display means, in this way he can know directly the steeping time for the tea.

The transport means is in the form of a strip and thus has a longitudinal extension along a longitudinal axis of the transport means, which is significantly greater than a transverse extension along a transverse axis of the transport means at right angles to the longitudinal axis. In each case, the longitudinal extension and the transverse extension are significantly greater than a thickness of the transport means at right angles to the longitudinal axis and to the transverse axis. A transport means in the form of a strip has the advantage that, with a small material requirement along the longitudinal axis, a relatively long-time region can be provided, in order to facilitate a precise measurement of time. In addition, in a plane spanned by the longitudinal axis and by the transverse axis, a display means relatively large in area can be provided, in order to facilitate reliable reading.

A transport means in the form of a strip also has the advantage that it can be manufactured and processed with ease. For example, a multiplicity of transport means in the form of a strip can be manufactured and processed together as a tape, from which the transport means can be cut out only at the end of a production process.

In a particularly simple embodiment, the transport means in the form of a strip is fundamentally rectangular. This produces the advantage in particular that a tape including a multiplicity of transport means can be cut into individual transport means free from waste.

A derived item of meta information is, for example, the reaching of a predetermined contact duration. If, for example, the reaching of an ideal steeping time or a given degree of infusion, for example “mild”, “balanced” or “strong”, is indicated, tea preparation becomes even easier in this, as the user himself does not have to know which steeping time equals a desired steeping result, so that incorrect operations are avoided.

Advantageously, the display means is optically freely accessible, so that it can be read off easily and unambiguously by a user. In particular, the display means is then optically freely accessible if, when using the time measurement apparatus, there are no or only transparent components of the time measurement apparatus in an optical path from the display means into an environment of the time measurement apparatus. In this way, a user can observe the display means from the environment unobstructedly.

As meant by embodiments of the invention, “tea” denotes an infusion drink that is prepared from various parts of different plants. In addition to types of tea that are in the main prepared from the leaves and leaf buds of the tea plant, this also includes in particular herbal teas and fruit teas.

The transport means includes

a) at least one reservoir region to accommodate liquid from an environment of the time measurement apparatus by an entry surface, arranged on an underside of the transport means,
b) at least one detection region for detection of the liquid and
c) at least one time region with one, two, three or more channels to transport the liquid in a defined transport duration from the reservoir region to the detection region.

The reservoir region has the advantageous effect that a larger amount of liquid can be stored here, so that an even and uninterrupted liquid feed into the time region is possible. The entry surface is advantageously designed for direct contact with the liquid. In this way, the liquid can penetrate into the reservoir region immediately during a bringing into contact with the liquid, so that measurement of the contact time can begin with a well-defined start point. The measurement is thus not imprecise or poorly reproducible, because the liquid must reach the entry surface initially, for example via a teabag.

Advantageously, the time region is designed so that the transport duration is significantly longer than a filling time for the reservoir region and a detection time, so that these are negligible compared with the transport duration. In this way, the contact duration, in which the liquid moves from the entry surface up to the detection region, is determined fundamentally by the transport duration, so that, by an appropriate design of the time region, in particular its length in the transport direction, it can be certain that the liquid reaches the detection region at the end of a contact duration determined precisely in advance.

The sensor is designed to detect the liquid in the detection region at an end of the time region which is remote from the reservoir region. This means that detection takes place at the end of a contact duration, which equals the sum of the filling time, transport duration and detection time and, for example, is set to the ideal steeping time for a tea. By a number of sensors or a single sensor with a number of places of detection, the end of different contact durations can be detected simply with a single time measurement apparatus.

The transport means comprises a combined system with an inner region for transport of the liquid and a moisture-repellent outer region, wherein the outer region encloses the inner region at a front surface of the transport means, at a rear surface of the transport means opposite the front surface, at a right-hand side edge of the transport means and at a left-hand side edge of the transport means opposite the right-hand side edge, at least in a section adjoining a lower edge for immersion in the liquid.

Surrounding the inner region with the outer region prevents liquid from entering the inner region in an uncontrolled manner through of the surfaces or side edges of the transport means during an immersion of the transport means in the liquid for measuring time and thus possibly measuring the time incorrectly.

In each case, the inner region is enclosed completely by the outer region at the surfaces and side edges, so that even if a user immerses the transport means in the liquid deeper than stipulated, no liquid can penetrate the inner region through the surfaces or side edges.

The inner region is freely accessible, at least at sections, at the lower edge and at an upper edge opposite the lower edge of the transport means. In particular, the inner region is not enclosed by the outer region at the lower edge and the upper edge. Since the inner region is freely accessible at least at sections at the lower edge, the liquid can penetrate the inner region through the entry surface arranged here in a controlled manner.

As the inner region is freely accessible at the upper edge, at least at sections, it is certain that air, which is displaced by liquid penetrating the inner region, can escape from the inner region and thus does not prevent transport of the liquid into the inner region.

This also produces the advantage that the upper edge and lower edge can be designed identically. In this way, the transport means can be manufactured in a particularly simple manner, for example by cutting a tape with a multiplicity of transport means arranged behind one another along their longitudinal axis into individual transport means, so that a single cut simultaneously produces a lower edge of a first transport means and an upper edge of a further transport means juxtaposed along the longitudinal axis to the first transport means.

In one embodiment, the inner region can be sealed at the upper edge in a moisture-repellent manner, for example by being enclosed by the outer region.

The time measurement apparatus can include at least one resistance element for limiting a flow of liquid from the reservoir region into the time region. The resistance element provides a defined flow into and consequently also within the time region. In this way, the period of transport for the liquid within the time region and thus also the measurement of time is particularly reproducible and precise.

Compared with the reservoir region and/or the time region, the resistance element can have a reduced cross-section for passage of the liquid and/or include a material with a reduced conductivity for the liquid and/or an increased material density. The resistance element is produced in a particularly simple manner by the named embodiments.

The inner region of the transport means can form the sensor and the display means in a particularly simple embodiment. In particular, the transport means can form the complete time measurement apparatus in this embodiment. For this, the inner region can for example consist of a material that changes at least one optical property, for example a color or its translucency, on contact with the liquid. In this way, a user can observe the progressive transport of the liquid in the transport means directly and, for example with the aid of at least one marking, in particular a time scale, read off the contact duration.

The inner region of the transport means has a translucency variable by contact with the liquid and is opaque in a dry state and translucent in a wet state. Opaque means that a marking arranged on a side of the inner region facing the rear surface does not show through the inner region and thus is virtually invisible for the human eye from the front surface. In the wet state, however, the inner region is translucent, so that the underlying marking can be perceived clearly from the front surface. In particular, “opaque” does not mean that the inner region is completely lightproof or that the marking could not shine through when backlit. “Translucent” does not mean that the inner region is completely transparent. On the contrary, what counts is whether the underlying marking is clearly perceptible through the inner region.

In particular, a transport means with variable translucency can be designed in the same way as the transport means described in the publication DE10151356A1.

By the variable translucency, the inner region can act in a particularly simple manner as sensor and display means, for example by the inner region, if it becomes wet by contact with liquid, releasing a view of a marking which symbolises a given contact duration.

In an embodiment with variable translucency of the inner region, it is unnecessary that a dye be transported by the liquid to indicate the contact duration, as in DE102015108921A1. By not using a transportable dye, in particular with an inner region freely accessible at the upper edge of the transport means, the risk of a contamination of the liquid outside the transport means by the dye is excluded. This is particularly advantageous if the liquid is a foodstuff, for example tea.

In addition, a transport speed of a dye by the liquid can be predicted only with difficulty, from which an inaccurate measurement of time can result. Moreover, the dye can smear during its transport in the transport means, so that the time measurement apparatus is not unambiguously readable for a user.

The transport means can be in the form of a strip or in the form of a thread. In this way, the transport means can be fitted in a simple manner to a teabag, in particular one usual in the art. In particular, the transport means can replace the thread of a teabag. By using a transport means in the form of a thread, which for example can be covered with a waterproof layer, the material cost compared with that for a transport means in the form of a strip is clearly reduced. Further advantages emerge in production, as a thread can be processed more easily in machines. Especially in comparison with paper strips, which must be inserted individually and have an increased handling cost. The lower material cost and handling cost result in a clear cost reduction.

The inner region of the transport means can consist of at least one absorbent material for the liquid. In this way, the liquid can be transported by capillary forces, in particular without further driving, for example a pump, within the transport means from the entry surface up to the detection region. The transport means can consist of at least one paper and/or at least one non-woven fabric. Paper, for example blotting paper, familiar from biotechnology, offers the advantages that it is biologically degradable and economical. A non-woven fabric offers the advantage that it can consist of synthetic materials, which have precise and predictable product properties, from which a particularly precise and reproducible measurement of time results.

In particular, the non-woven fabric can be heat-sealable, for example by an adequately high thermoplastic material share of at least 30%, in particular at least 50%, and/or biologically degradable, for example consisting of polylactides (PLA). A paper can also be heat-sealable, for example by a thermoplastic material contained in it or applied to it, for example consisting of polylactides. Heat-sealable materials can be connected by heat, for example by hot lamination or welding, in particular ultrasound welding, without an additional adhesive forming a material bond between them.

If the inner region is heat-sealable, this produces the advantage that the inner region can be connected to form a material bond with the outer region without an additional adhesive, which, in particular, can be problematic for applications in the food industry, in order to guarantee a high stability of the transport means and thus a reliable measurement of time. Furthermore, the inner region can be fixed to the outer region by a material bond over a large area, in particular the entire area, so that a marking applied to the outer region is particularly clearly recognisable through the inner region, if this is translucent due to contact with the liquid.

Advantageously, the reservoir region, the transport region, the detection region and in particular also the resistance element have the same composition, and/or the transport means is in one piece. These embodiments allow a particularly simple construction of the time measurement apparatus.

Advantageously, the transport means comprises an inner region to transport the liquid and a moisture-repellent outer region enclosing the inner region at least in the time region and in the detection area. Advantageously, the outer region encloses the inner region also in the reservoir region except for the entry surface. “Moisture-repellent” as meant by embodiments of the invention means, on one hand, that during a typical storage duration, especially for tea, under typical storage conditions, especially for tea, no amount of moisture penetrates the transport that is so great that a subsequent time measurement is mistaken, for example because the sensor detects liquid too early, caused by moisture absorbed earlier during storage in the transport medium. “Moisture-repellent” as meant by embodiments of the invention means, on the other hand, that during use, within a typical usage duration, especially a steeping time for a tea, no amount of liquid that could falsify the time measurement penetrates into the transport means, other than through the entry surface. A particularly reliable time measurement is therefore achieved thanks to the outer region. In particular, a sensitive sensor can be used thanks to the outer region, without the danger of a false-positive detection.

The outer region comprises at least one moisture-repellent support, a cardboard coated with a thermoplastic synthetic material, in particular a polylactide, for stiffening the transport means. Even with a very thin inner region, the support can lend the transport means an adequate mechanical stability. A very thin inner region is advantageous, because the transport of the liquid substantially takes place in one plane and is therefore predictable in a particularly reliable manner. Also, a thin inner region can be enclosed in a moisture-repellent manner particularly easily without, for example, air inclusions occurring between the inner region and the outer region, which could influence the liquid transport in an uncontrolled manner.

By the support, the transport means can, for example, be placed upright in a drinking cup, so that the transport of the liquid in it can be read off with ease and the open upper edge of the transport means does not come into contact with the liquid. Furthermore, an adequately stiff transport means can also be used to stir the liquid.

The transport means can be stiffened by a connection between inner region and outer region, for example a welded seam and/or a number of spot welds.

The transport means can be stiffened by at least one bend or fold, fundamentally parallel to the side edges.

The support is elastically bendable, so that without a risk of damaging it, it can be processed by machine and stored in a space-saving manner, for example bent around a teabag, and sets back for use in a straight form by itself.

The support can have at least one defined bending region, in which it can be bent more easily than outside the bending region. This ensures that the support, for example if it is packed, bends primarily in the bending region, which can, for example, be outside the time region, so that the time measurement is not distorted by a bending of the transport region.

By a coating with a thermoplastic synthetic material on at least one side, on both sides, an economical, easy to process and biologically degradable material, such as a cardboard, can be designed to be moisture-repellent. In addition, a thermoplastic synthetic material is heat-sealable, so that the appropriately coated cardboard can be connected without an additional adhesive to form a material bond with other components of the time measurement apparatus or, for example, a teabag.

The outer region can comprise at least one moisture-repellent non-woven fabric. The inner region can consist of a non-woven fabric absorbent for the liquid. In particular, the transport means can consist of a multi-layer non-woven fabric, for example with two moisture-repellent layers and a layer of absorbent non-woven fabric arranged between them.

The outer region can comprise at least one surface impregnation or coating of the inner region. An impregnation of the surface, for example by spraying, can make the surface just as waterproof as the use of a film, but is fundamentally faster and simpler to do and demands fewer expensive machines than a weld in a film. Advantageously, the impregnation or coating in is applied in liquid form, for example by immersion in a bath or spraying.

The outer region can comprise at least one moisture-repellent and, at least in sections, transparent film, made from a thermoplastic synthetic material. The outer region can be designed like the coating described in DE102015108921A1.

The outer region can comprise at least one air outlet to extract at least air from the inner region. The air outlet is advantageously arranged at the far end of the transport means from the entry surface. The air outlet prevents the air enclosed in the transport means hindering an even transport of the liquid from the entry surface to the detection region. The air outlet can be closed with a membrane, which lets gases pass selectively and holds liquids back. This prevents liquid entering or leaving through the air outlet, which might distort the time measurement or impair the handling of the time measurement apparatus, for example because a user gets wet fingers if he grasps the time measurement apparatus in the region of the air outlet.

The outer region can be modified thermally or chemically compared with the inner region. For example, a transport means consisting of an absorbent material can be sealed by heat treatment to make it moisture-repellent in the outer region.

The outer region can be translucent, in particular transparent, at least in sections, in particular in the display means region. By a translucent outer region, at least in sections, the transport means can be protected, and the display means can be optically freely accessible, so that a clear and thus reliable legibility of the display means is guaranteed.

The time measurement apparatus can comprise at least one closing device for temporary closure of the entry surface against a penetration of moisture into the reservoir region. “Temporary” means here in particular that the entry surface is closed during a storage of the time measurement apparatus and can be opened during a use of the time measurement apparatus. This way the transport means can be protected against moisture until the time measurement apparatus is used, whereby, even after protracted storage a reliable time measurement is possible. The closure device can, for example, comprise a film covering the entry surface, which can be removed before using the time measurement apparatus, for example by tearing it off.

The closure device is advantageously soluble in the liquid. This simplifies the use of the time measurement apparatus, as the closing device does not have to be opened manually. The closure device can, for example, consist of a material that dissolves in the liquid, for example a sugar.

In particular, the closure device can be soluble in the liquid only above a minimum temperature. The minimum temperature can, for example, be the same as a minimum steeping temperature for a tea. This way a user, for example at the closure device, can detect in a simple way that the water used by him for brewing the tea is not hot enough. The closure device can, for example, comprise a candied sugar, which dissolves only above the minimum temperature on a use-related time scale, for example of one or a few minutes, if a tea steeping time is to be measured.

In a particularly advantageous embodiment, the rear surface of the transport means is formed by the support and the front surface of the transport means is formed by the film, and the inner region of the transport means is arranged between the support and the film. By this particularly simple construction as a three-layer combined system, on one hand a good mechanical stability of the transport means and on the other hand a good visibility of the inner region and thus a good legibility of the contact time through the film is guaranteed. In addition, the inner region can be enclosed by the support and the film in a moisture-repellent way.

In each case the support and the film form an overhang over the inner region at the side edges of the transport means, wherein at least the support and the film are interconnected at the side edges in a moisture-repellent manner, in particular forming a material bond. With the aid of the overhang, the inner region can be enclosed at the side edges particularly simply and reliably in a moisture-repellent manner.

In an embodiment, the support and the film can be closed flush with the inner region at the side edges without an overhang. This embodiment allows a particularly simple productionbecause support, film and inner region can have the same format and thus, for example, can be cut out together from a prefabricated combined system. However, a disadvantage in this embodiment is that a moisture-repellent sealing of the side edges is more expensive.

The inner region and the support are interconnected, at least in sections, to form a material bond. This ensures that the inner region rests on the support, at least in the detection region, so that a marking applied to the support is clearly legible through the inner region, if this is translucent due to contact with the liquid. In particular, this prevents air or liquid accumulating between the inner region and the support, which could impair legibility.

The display means comprises a marking, in particular a colored marking, wherein the marking is applied at a side of the inner region facing the rear surface on the inner region and/or on the outer region, so that the marking is visible in a wet state of the inner region through the inner region from the front surface.

The marking is separated from the inner region by a moisture-repellent material, in particular a moisture-repellent film and/or a moisture-repellent coating of a support. This ensures that the liquid does not come into direct contact with the marking, so that a contamination of the liquid, for example by a dye from the marking, is excluded. In particular then, if the liquid is a foodstuff, for example tea, exclusion of contaminations is particularly important.

The marking can, for example, be printed on the support, before or after this is provided with a moisture-repellent coating.

The time measurement apparatus can comprise a multiplicity of channels to transport the liquid within a number of different transport durations from the reservoir region to the detection region. By a multiplicity of channels, the end of different contact times can be detected with a single time measurement apparatus, in particular with a single sensor. This way, for example, the ideal steeping time for different teas and/or for different infusion grades can be detected in each case.

The channels can be connected in a liquid-conducting manner with the reservoir region by resistance elements different from each other by a maximum flow of liquid from the reservoir region into the relevant channel. This way the flow through the channels and thus the transport duration in each case can be adjusted.

The channels can have paths of different lengths and/or flow resistances for the flow from the reservoir region to the detection region. This way the transport duration through the channels can be adjusted in each case.

The channels can have a number of closure devices for temporary closure of a channel in each case against a transport of liquid from the reservoir region to the detection region, wherein the closure devices in particular can be soluble in the liquid. In particular, the closure devices can be soluble at different temperatures, so that channels with different transport durations are released according to temperature. This way, for example, depending on the temperature of the liquid, the end of contact times differing in length can be detected, for example to detect the end of an ideal steeping time differing in length, depending on the temperature of the infusion water.

The channels can be connected for conducting liquid with a multiplicity of reservoir regions, which comprise particularly in each case a closure device for temporary closure of the entry surface of the relevant reservoir region against a penetration by moisture. By connecting with reservoir regions with different closure devices, the same effect can be achieved as described in the preceding paragraph for different closure devices on the channels.

The at least one sensor and the at least one display means can be formed by at least one dye arranged on and/or in the transport means. The dye can, for example, be arranged in the time region and be transportable by the liquid into the detection region. The dye can, for example, be variable in color by contact with the liquid. The liquid can thus, if it is transported through the transport means, take the dye with it on its path and/or change the color of the dye, for example by the pH value of the liquid. In each case, the user can obtain a good view of the course of the liquid in the transport means using the dye. In particular, the dye can be designed as described in DE102015108921A1.

It is conceivable to use dyes that only change their color or become visible by contact with the liquid after a certain contact time. These dyes are activated, for example, if the dye comes into contact with a set temperature (e.g. starting from 80° C.) or the liquid (e.g. the water for the tea). With such dyes, written markings (e.g. “mild”, “OK” and “strong”) could be applied, that become visible only if the dye comes into contact with the liquid at the site of the relevant written marking, e.g. after a contact time of 3 min, 4 min or 5 min. Because of this, it is simpler for the user to understand whether the desired steeping time was already reached.

It is conceivable, to make the dye visible only if a certain contact time (e.g. 2 minutes for green tea) is reached. This can be reached by overlaying or covering the transport means in part, especially in the time region, with an opaque material. The opaque material can, for example, comprise a film, which is transparent only at points which the liquid reaches after a pre-set time (e.g. after 2 minutes for green tea). The advantage for the user is that he is notified visually only when the steeping time has been reached. Prior to this, there is no distraction.

The display means can comprise at least one marking, in particular a number of markings, for example in the form of a time scale. Alternatively, or complementing a time scale, a scale related to the degree of infusion of a tea is advantageously also conceivable, for example “mild”—“balanced”—“strong”. By the at least one marking, the user can read off directly and simply from the path the contact time or the end of a given contact time.

The at least one marking can be applied to the transport means, in particular at an outer region of the transport means. If the marking is applied directly to the transport means, a user can compare the path travelled by the liquid in the transport means particularly simply and precisely with the position of the marking to read off the contact time.

The time measurement apparatus can comprise at least one restraining means to hold back the dye in the time measurement apparatus. By the restraining means, an escape of the dye into the liquid, problematic for example under food legislation, can be prevented. The restraining means can, for example, comprise a suction reservoir, which is arranged downstream of the detection region in the direction of transport of the liquid within the transport means. The suction reservoir ensures that, at least over a typical usage duration of the time measurement apparatus, there is at all times a liquid transport from the entry surface through the transport means into the suction reservoir, and no liquid, possibly with dye dissolved in it, escapes from the time measurement apparatus. The restraining means can comprise a chemical bonding of the dye to the transport means and/or a membrane impermeable to the dye at the entry surface and, in particular, also at an air outlet.

Advantageously, the transport means, in particular the entire time measurement apparatus, is biodegradable and compostable. As meant by embodiments of the invention, “biologically degradable” means at least a restricted biodegradable capability in accordance with the tests of the OECD test series 302 (A-C), a fast and complete breakdown under aerobic conditions in accordance with the OECD tests series 301 (A-F). “Compostable” means at least a degradable capability of 60% within 180 days in accordance with the American standard ASTM D-6400, in particular a conversion of at least 90% into CO₂ in 6 months in accordance with the European standard EN 13432.

Thanks to biological degradability, the transport means can, for example, be broken down biologically together with a teabag fixed to it. Biological degradability can, for example, be achieved by producing the transport means, in particular the complete time measurement apparatus, from paper and biologically degradable synthetic materials, for example polylactides.

The at least one sensor can comprise an electronic sensor, in particular at least one moisture sensor, particularly with a number of electrodes for contact with the liquid, and/or at least one optical sensor for optical detection of the liquid, a dye and/or a color change of the dye. The electrodes can, for example, be printed on the transport means for a production of the time measurement apparatus that is as simple as possible. The optical sensor can, for example, be a camera, in particular a camera integrated into a computer device, for example a smartphone.

By an electronic sensor, the liquid can be detected particularly precisely and the output signal from an electronic sensor can be further processed in many different ways. In particular, a time measurement device with an electronic sensor can be formed even without transport means, if the electronic sensor is designed for the purpose of starting an electronic contact time measurement on contact with the liquid.

The at least one display means can comprise at least one electronic signal output device for outputting at least one signal, a vibration signal, acoustic signal and/or optical signal. By the electronic signal output device, the user of the time measurement apparatus can be notified particularly clearly, in particular via several senses, of the measured contact time or the end of a pre-set contact time. The display means and the sensor can be separated in space from one another and, in particular, be interconnected only communicatively, for example via near field communication (NFC) or Bluetooth. The display means can, for example, be integrated in a computer device, in particular a smartphone.

The time measurement apparatus can comprise at least an energy supply, in the simplest case with a connection for an external energy source, to supply the at least one sensor and/or at least one display means with energy. The energy supply can comprise at least an energy store, for example a battery or an accumulator, printed on the transport means, and/or an energy recovery device, for example a Peltier element or a photovoltaic module. By an energy store, especially in combination with an energy recovery device, the energy supply is advantageously independent of an external energy source, so that the time measurement apparatus can be used anywhere.

The time measurement apparatus can comprise at least one communication device connected communicatively with the at least one sensor for communication, in particular wireless communication, with a computer device, for example a smartphone. The communication device can comprise, for example, a near field communication- and/or Bluetooth sender-receiver. Advantageously, by the communication device, sensor measuring data, for example for analysis, storage and/or display, can be transferred to the computer device.

The time measurement apparatus can comprise at least one data memory for storage of data calculated by the sensor, meta information derived from it and/or reference data.

The time measurement apparatus can comprise at least one identification means for identification of the time measurement apparatus, wherein the identification means comprises an alphanumeric code, a barcode, a QR code and/or a RFID transponder.

The time measurement apparatus can comprise at least one temperature display to display a liquid temperature, wherein the temperature display comprises a liquid crystal thermometer. By a temperature display, a user of the time measurement apparatus can, for example, check that the infusion water has the right temperature for preparation of a tea. A liquid crystal thermometer has the advantages that it is flexible, particularly flat and constructed without moving parts, so that it can be integrated simply and economically in the time measurement apparatus.

By the aforementioned electronic components, in addition to visual perception further sensory organs can be addressed. Inter alia, other sensory organs can be addressed by tones, vibration and/or light at a reached and/or desired steeping time. The electronic components can, in particular, replace completely or only complement the transport means. It is, for example, possible to apply conducting tracks in different printed versions on paper, in particular on the transport means. For this purpose, batteries, LEDs, microcontrollers, wireless chips, sensors (inter alia moisture sensors) and loudspeakers exist, which can be applied on paper. It is, for example, possible to measure the rising liquid in the transport means at various path lengths with a moisture sensor and then, for example, output a different tone according to the pathlength.

Some new options are generated by the quoted electronic extensions. Inter alia, it is possible to play back a tone, output a light signal and/or a wireless signal (e.g. by Bluetooth) to a computer device, in particular a mobile telephone. This signal can then, e.g. be received by an app, in order to run a very wide variety of functions. In sum, the probability that the steeping time will be met is increased by building in electronic components.

Further electronic components could also be RFID chips. This way customisation of the time measurement apparatus can become possible. Unambiguous identification (e.g. as serial numbers), for example for a traceability and a customer retention (e.g. administration of previously consumed products, which were prepared with the time measurement apparatus), can also be provided.

A filter paper web according to embodiments of the invention for producing a multiplicity of, in particular pyramidal, teabags is characterised in that a time measurement apparatus according to embodiments of the invention is fixed on the filter paper web for each teabag. By applying the time measurement apparatus on the filter paper web, a teabag with a time measurement apparatus applied to it can be produced in a particularly simple manner, especially without modifying the process for producing the teabag from the filter paper web.

The filter paper web is particularly advantageous for producing pyramidal teabags, because such teabags are usually produced already from a filter paper web with teabag threads applied to it. Thus, only the threads have to be replaced by time measurement apparatuses, without modifying the production process further.

The rear face of the transport the time measurement apparatus is fastened at least by at least one lower fastening near the lower edge of the transport means on the filter paper web. A fastening on the rear face has the advantage that reading off the contact time on the front face is not obstructed by the teabag.

The fastening can, for example, comprise a material bond, in particular a heat-seal connection. Usually, filter paper webs for teabag production are heat-sealable, for example because they are produced from polylactides. According to embodiments of the invention, the rear face of the transport means can also be heat-sealable, for example by a thermoplastic coating of a support of the transport means.

At least one fastening is provided near the lower edge of the transport means, where the entry surface for the liquid can be located. This ensures that the entry surface dips into the liquid together with the teabag, so that the entry surface comes into contact with the liquid simultaneously with the teabag and thus the start times of the contact duration of time measurement apparatus and teabag with the liquid are synchronised.

The rear face of the time measurement apparatus transport means is fastened at least by at least one upper fastening near the upper edge of the transport means on the filter paper web. The additional upper fastening ensures that the transport means is not removed from the teabag during processing of the filter paper web to teabags or during further processing or the transport of the teabag. In addition, the transport means can be bent around the teabag in a space-saving manner by at least two fastenings during production of the teabag.

The upper fastening is designed to be less strong than the lower fastening, for example perforated and/or with a smaller contact surface, so that the upper fastening can be released by a user of the teabag. In this way, for example, a transport means bent around the teabag by upper and lower fastenings can be released at one side from the teabag and bent straight, by elastic recovery of the transport means, for using the teabag and measuring the steeping time. In the straight state of the transport means, the steeping time can be measured and read off reliably. In addition, the transport means can also be used to hold the teabag, for which purpose the teabag thread is usually used. Accordingly, the transport means can replace the teabag thread, whereby production of the teabag is simplified.

The filter paper web can be foldable about a longitudinal axis of the filter paper web for production of the teabag in a central folding region of the filter paper web, wherein the time measurement apparatus transport means is arranged transversally to the longitudinal axis on the filter paper web and fastened to the filter paper web by a central fastening, in particular between the lower edge of the transport means and the central folding region.

The transport means is held particularly reliably on the teabag by an additional central fastening. The central fastening is designed to be stronger than the upper fastening, so that only the upper fastening is released by a user of the teabag. The remaining central and lower fastening can then be used together with the teabag fastened to it, for example to stir the tea.

The central fastening is arranged between the lower edge of the transport means and the central folding region, in order that the lower and central fastenings are on the same side face of the finished produced teabag, so that the transport means is not turned or bent by the lower and upper fastenings.

A time measurement system according to embodiments of the invention with a, in particular pyramidal, teabag is characterised in that a time measurement apparatus according to embodiments of the invention is fastened to the teabag. The rear face of the transport the time measurement apparatus is fastened to the teabag at least by a lower fastening near the lower edge of the transport means on the tea bag. From this arise the advantages and design options described previously for the filter paper web according to embodiments of the invention. In addition, the transport means can be fastened to the teabag by one or more additional fastenings as described for the filter paper web, from which arise the advantages and design options described earlier.

A time measurement system according to embodiments of the invention comprises at least one-time measurement apparatus according to embodiments of the invention. The time measurement apparatus is integrated at least in part in a vessel, a drinking vessel, and/or a stirrer, a spoon, for the liquid and/or a tea container, a teabag, a tea filter, or a tea stick.

Advantageously, for example, relatively expensive components of the time measurement apparatus, usable many times, such as an electronic sensor or an electronic display device can be integrated in a vessel that is usable many times, for example a teapot or a teacup, or a stirrer that can be used many times. In this way, the costs for using the time measurement system can be reduced and resources for production saved.

Advantageously, the time measurement apparatus can be fastened to a tea container, in particular a standard teabag. This ensures that the time measurement apparatus is immersed in the infusion water simultaneously with the tea container, so that the measured contact time is the same as the tea steeping time. In particular, the time measurement apparatus can replace a teabag thread. This way the application of the time measurement apparatus can be integrated into teabag production particularly easily.

A time measurement apparatus fastened to a tea container can have an increased stiffness compared with a teabag thread and/or, in particular near the entry opening, a ballast weight. By an increased stiffness, the time measurement apparatus can advantageously be used to stir the tea. A ballast weight ensures that the entry surface dips completely into the infusion water, so that the infusion water can reach the transport means through the entry surface.

The tea container can comprise a squeezing device and/or a holding device for the tea container. By a squeezing device, for example a folding flap on the time measurement apparatus, the tea container can be squeezed at the end of the steeping time, in order to empty or dispose of it without uncontrolled drips. By a holding device, for example with a number of flaps and/or hooks on the time measurement apparatus, the tea container can be held securely in a vessel for tea preparation.

A time measurement method according to embodiments of the invention is executed with a time measurement apparatus according to embodiments of the invention and comprises at least the following steps:

a) Bringing the entry surface of the reservoir region of the transport medium of the time measurement apparatus into contact with a liquid at a contact start time,
b) Transporting the liquid through the time region of the transport medium within a transport duration into the detection region of the transport medium,
c) Detecting the liquid in the detection region at a detection time, which is correlated with the contact start time, and
d) Displaying the contact duration or an item of meta information derived from the contact time.

Advantageously, the steps are executed in the sequence quoted.

Advantageously, the bringing into contact takes place directly, in other words especially not via a further medium, for example a teabag, between entry surface and liquid, so that no poorly controllable delay caused by a transport of the liquid through the further medium occurs.

In an alternative embodiment, transporting can be waived, if an electronic contact time measurement is started, for example by the contact of an electronic sensor at the contact start time.

Advantageously, the detection time follows up to negligible deviations by the transport duration on the contact start time.

An item of meta information is, for example, the end of a pre-set contact duration, for example an ideal tea steeping time. In addition to the contact duration, further information, for example a liquid temperature, can also enter into the item of meta information. Thus, for example, depending on the temperature of the infusion water, the end of another ideal steeping time for a tea can be displayed.

The step of detecting can comprise an optical, visual, detection of a change of translucency of the inner region of the transport the time measurement apparatus caused by the liquid. In particular, the inner region can become transparent by contact with the liquid, so that a marking applied to the side of the inner region facing the rear surface of the transport means becomes visible through the inner region from the front surface of the transport means.

The step of detecting can comprise an optical, visual, detection of the liquid, in particular a dye transported by the liquid into the detection region and/or a color change of a dye applied in the detection region caused by the liquid. A visual detection can take place particularly simply and in particular without further aids with a particularly simply constructed time measurement apparatus. A visual detection can take place in particular as described in DE 102015108921A1, which are included here by reference.

The step of detecting can comprise an electronic detection of the liquid, with a moisture sensor. An electronic detection offers the advantages of high precision and reliability and also opens a host of options for further processing of the detection signal.

The step of bringing into contact can comprise opening of a closing device for temporary closure of the entry surface against a penetration of moisture into the reservoir region by the liquid. In particular, opening can take place only above a given temperature, for example a minimum infusion temperature for a tea.

The step of displaying can comprise an output of at least one signal, a vibration signal, wireless signal, acoustic signal and/or optical signal. The wireless signal can, for example, be transmitted to a computer device, in particular a smartphone, for example for displaying the contact duration on the computer device.

A production method according to embodiments of the invention is used to produce a time measurement apparatus according to embodiments of the invention and, in particular, a time measurement system according to embodiments of the invention and comprises at least the following steps:

a) Provision of a blank for an inner region to transport the liquid,
b) Application of a section of a moisture-repellent outer region at least to a front surface and a rear surface opposite the front surface of the blank,
c) Whereby the sections form in each case an overhang over the blank at a right-hand side edge of the blank and a left-hand side edge of the blank opposite the right-hand side edge, and
c) Pressing together at least the overhangs for moisture-repellent connection of the sections with each other.

Advantageously, the steps are executed in the sequence quoted.

The blank is advantageously in the form of a strip and can have in its plane a homogeneous material composition or a structured material composition. For example, the blank can comprise suction-capable regions for the liquid, in particular strips, for forming in each case an inner region and moisture-repellent regions, in particular strips, for forming a part of the outer region. By a structured material composition, in the further method of production, a moisture-repellent outer region enclosing the inner region can be formed particularly easily. The blank consists, for example, of at least one non-woven fabric.

The applied sections can be identical or different from one another as regards their material properties. For example, one section can be at least partially transparent and the other section opaque. The sections can comprise at least one non-woven fabric. An at least partially transparent section can comprise a film.

In particular, the section applied on the front surface can comprise a moisture-repellent film, at least transparent in sections, and the section applied on the rear surface can comprise a moisture-repellent support for stiffening the transport means.

In an embodiment of the method, wherein the sections do not form an overhang over the inner region, the method comprises a compression of the sections at least at a right-hand and left-hand edge of the blank.

By the compression, the sections are advantageously brought together so closely that, between them, during a normal duration of use of the time measurement apparatus, no volume of liquid that could influence the time measurement penetrates the inner region. It is therefore particularly advantageous if the blank is moisture-repellent where the sections are compressed.

The compression can comprise a weld, in particular an ultrasound weld, and/or a lamination, in particular a hot lamination or heat sealing, of the sections with one another and of at least one section with the blank. By a weld or lamination, a moisture-repellent and permanent connection can be achieved with established methods and without additional components, for example an adhesive. An ultrasound weld offers the particular advantage that only a small thermal loading occurs in the environment of the welding and even relatively thick combined systems can be welded over their entire thickness.

By an ultrasound weld, a pattern of fixing points fixing the sections to the inner region can be produced. By the density and distribution of the fixing points, for example, a flow of the liquid within the inner region, the visibility of a marking through the inner region and/or the mechanical stiffness of the transport means can be controlled locally. In addition, such a pattern can represent a feature for security of the transport means against forgery.

For example, a low density of fixing points can be stipulated in a reservoir region and a bending region of the transport means, in order to guarantee a high flow and a high flexibility. However, a high number of points can be stipulated in a detection region, in order on one hand to guarantee a precise legibility and on the other hand to achieve a close contact of the inner region with the outer region and thus a good detectability of a marking on the outer region through the inner region.

A heat seal or hot lamination offers the advantage that, thereby, large surfaces can be connected quickly and economically over their full area.

Compression can comprise an imprinting in the transport an item of information, in particular a marking or an identification, in particular to improve security against forgery. Imprinting produces the advantages that neither an additional step to the method nor an additional dye is needed to apply the information.

The production method can comprise a connection of the transport means with a tea container, in particular by a weld, for example an ultrasound weld. The tea container is advantageously a teabag, in particular a pyramidal teabag.

The production method can comprise an application of a dye to the blank, before applying the second section.

The production method can comprise a cutting of the transport means, in particular after compression. By cutting the transport means only after compression, a multitude of transport means can be prepared in parallel in the previous steps, so that a particularly fast and economical production is possible.

### BRIEF DESCRIPTION

Some of the embodiments will be described in detail, with reference to the following figures, wherein like designations denote like members, wherein:

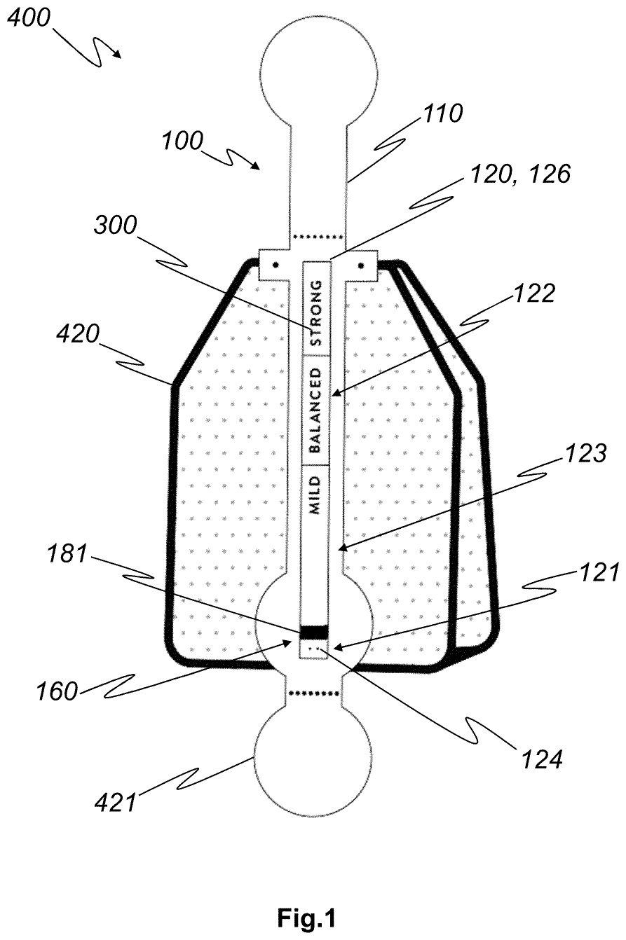

FIG. 1 shows a schematic illustration of a time measurement system according to embodiments of the invention;

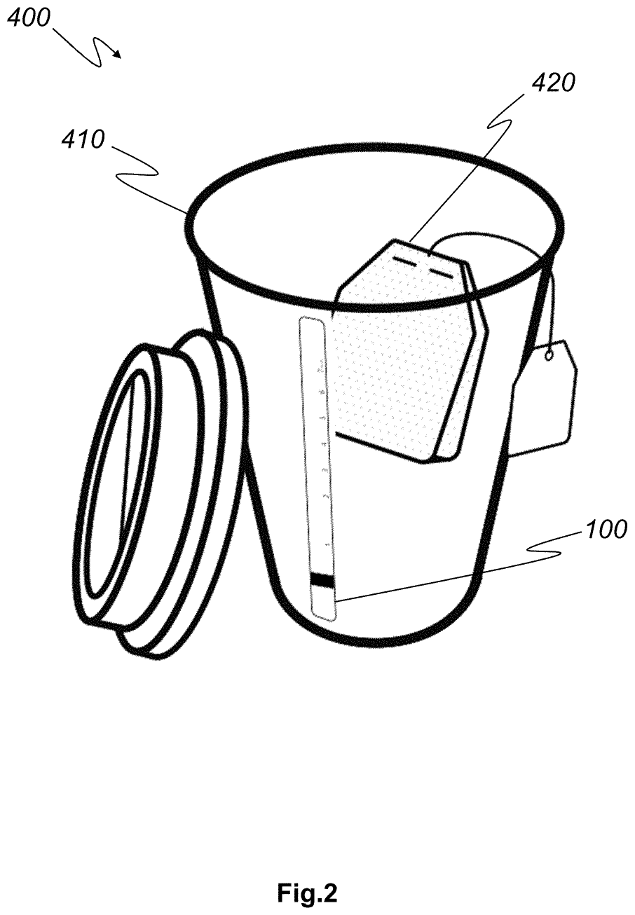

FIG. 2 shows a schematic illustration of a further time measurement system according to embodiments of the invention;

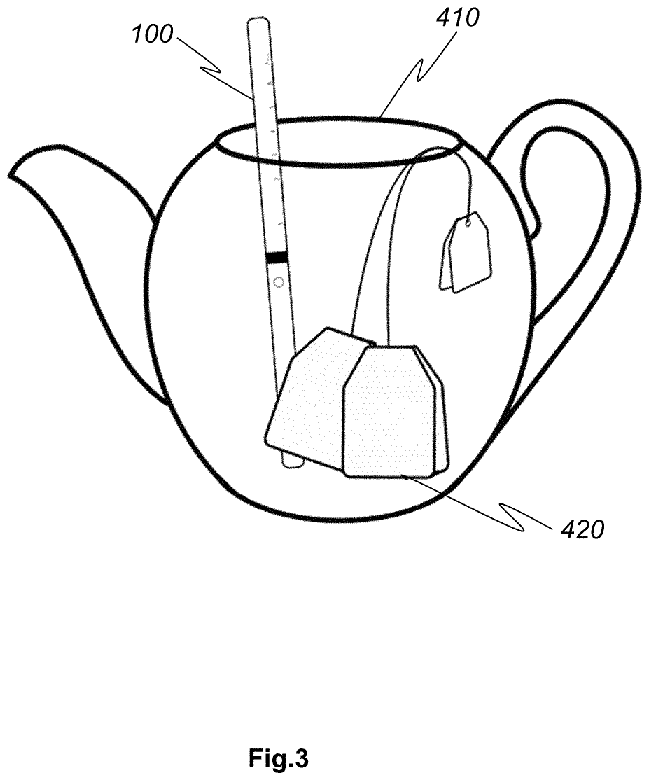

FIG. 3 shows a schematic illustration of a time measurement apparatus according to embodiments of the invention;

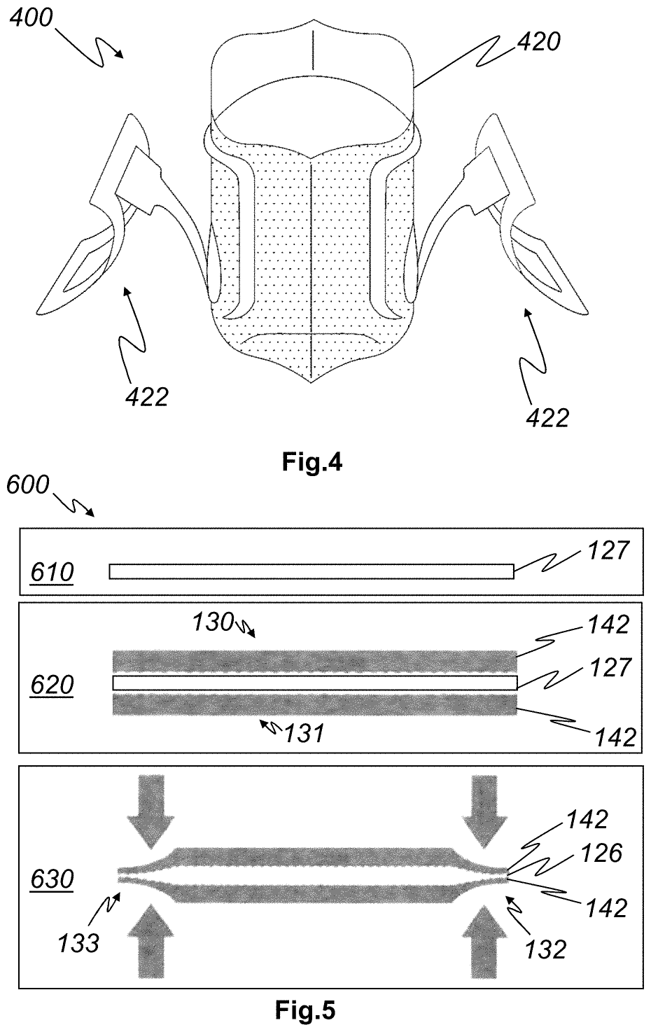

FIG. 4 shows a schematic illustration of a further time measurement system according to embodiments of the invention;

FIG. 5 shows a schematic illustration of a production method according to embodiments of the invention;

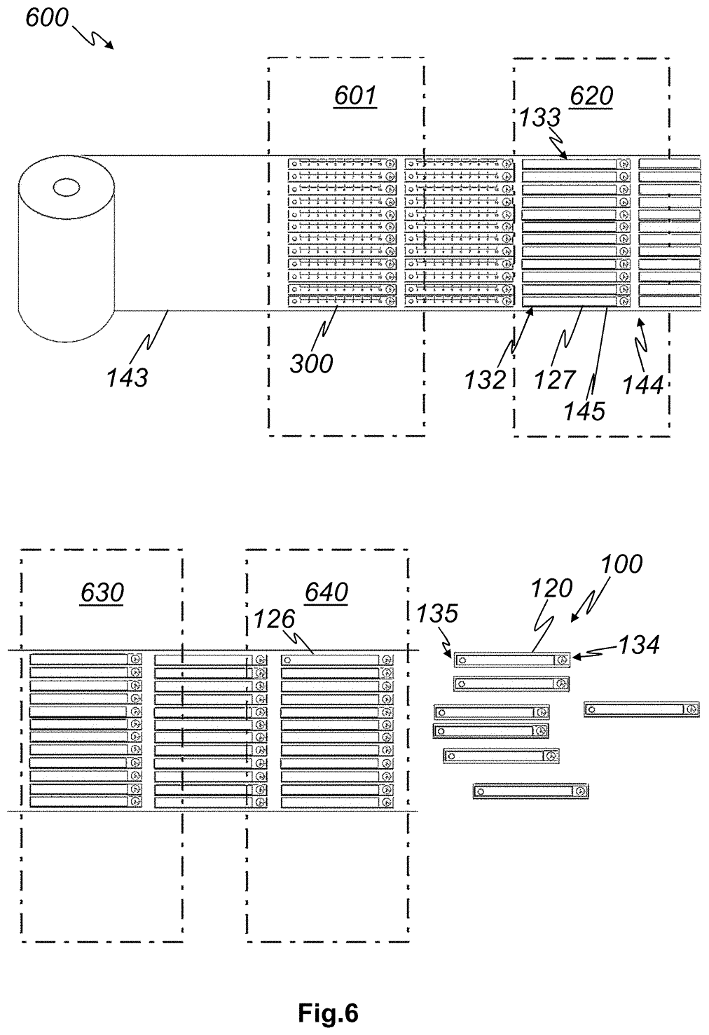

FIG. 6 shows a schematic illustration of a further production method according to embodiments of the invention; and

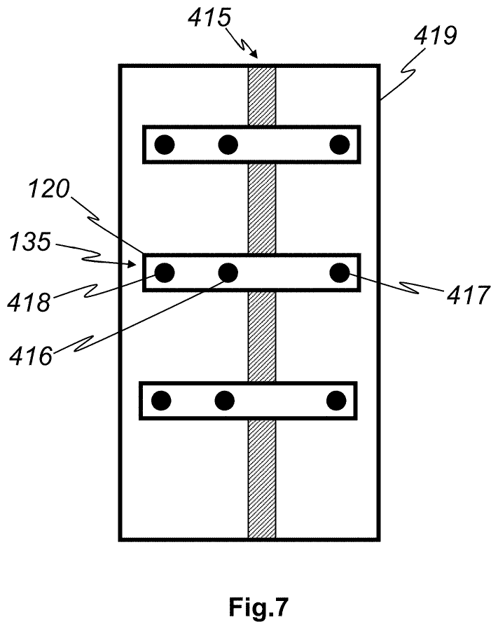

FIG. 7 shows a schematic illustration of a filter paper web according to embodiments of the invention.

### DETAILED DESCRIPTION

FIG. 1 shows a schematic illustration of a time measurement system 400 according to embodiments of the invention. The time measurement system 400 illustrated comprises a teabag 420 and a time measurement apparatus 100 according to embodiments of the invention fastened to it, for example welded to it. The time measurement apparatus 100 comprises a transport means 120 for a liquid, which comprises a reservoir region 121 with at least one entry surface 124 for the liquid, a time region 123 and a detection region 122. The transport means 120 comprises an inner region 126, for example a strip of absorbent paper or non-woven fabric, to transport the liquid and a moisture-repellent outer region (not illustrated), for example a film or a moisture-repellent non-woven fabric. The outer region can comprise at the entry surface 124 at least one opening for the liquid. On the inner region 126, for example, a dye 181 is applied, which forms a sensor and a display means.

If the time measurement system 400 is immersed with the entry surface 124 in a liquid, for example hot water for brewing the tea, the liquid can penetrate into the inner region 126 through the entry surface 124 and is transported by capillary forces within the inner region 126 from the reservoir region 121 through the time region 123 into the detection region 122. At the same time, the dye 181 can be carried along by the liquid. The detection region 122 comprises a number of, for example three, markings 300 (“MILD”, “BALANCED”, “STRONG”). If the liquid with the dye 181 reaches the markings 300, a user can detect from this that a contact time with the liquid which, for example, corresponds to a degree of infusion of the tea represented by the relevant marking 300, has elapsed.

In the path of the liquid from the reservoir region 121 into the time region 123, a resistance element 160, for example a reduced flow cross-section, can be arranged to limit the flow.

A section of the outer region of the transport means 120 can comprise a support 143, made for example from a cardboard, in particular resistant to liquid, and, in particular, be connected via this with the tea container 420. The time measurement system 400 can comprise, formed in particular as a folding flap of the support 143, a squeezing device 421 for the teabag 420.

FIG. 2 shows a schematic illustration of a further time measurement system 400 according to embodiments of the invention. In this case, a time measurement apparatus 100 according to embodiments of the invention is applied to a lateral wall of a vessel 410, in particular of a tea mug. In addition, the vessel 410 can contain a tea bag 420.

FIG. 3 shows a schematic illustration of a time measurement apparatus 100 according to embodiments of the invention which, for use, is immersed together with a number of, for example two, teabags 420 in a liquid (not illustrated), for example hot infusion water, in a vessel 410, for example a teapot.

FIG. 4 shows a schematic illustration of a further time measurement system 400 according to embodiments of the invention with a teabag 420, in particular open at its upper side, and a time measurement apparatus according to embodiments of the invention applied to it (not illustrated). The teabag 420 comprises a number of, for example two, holding devices 422, for example folding cardboard flaps, for fastening the teabag 420, for example to a teacup (not illustrated).

FIG. 5 shows a schematic illustration of a production method 600 according to embodiments of the invention for a time measurement apparatus 100 according to embodiments of the invention. The production method 600 comprises a provision 610 of a blank 127, for example an absorbent non-woven fabric in the form of a plane, for an inner region to transport the liquid.

After the provision 610, an application 620 takes place of a section 142 of a moisture-repellent outer region 140, for example a moisture-repellent non-woven fabric, at least on a front surface 130 and a rear surface 131 opposite the front surface 130 of the blank 127.

After the application 620, a compression 630 takes place (represented by arrows), for example an ultrasound weld, of the sections 142 at a right-hand side edge 132 of the inner region 126 between the front surface 130 and the rear surface 131 and at a left-hand side edge 133 of the inner region 126 opposite the right-hand side edge 132.

FIG. 6 shows a schematic illustration of a further production method 600 according to embodiments of the invention for a time measurement apparatus 100 according to embodiments of the invention. The production method 600 comprises a printing 601 on a support 143, for example a cardboard web coated with a thermoplastic material, in particular with polylactides, in particular with a thickness of 1 mm to 2 mm, of a number of markings 300, for example timescales, wherein a multiplicity of markings are printed on the support 143 parallel to one another.

After the printing 601, an application 620 takes place of a blank 127 for an inner region 126 of the transport means 100, for example a strip of absorbent paper, in particular with a thickness of 25 μm to 50 μm, on each of the markings 300. In addition, an application 620 of a moisture-repellent film 144, at least transparent in sections, for example made from polylactides, in particular with a thickness of 50 μm to 100 μm, is made on the blanks 127.

The application 620 of the blanks 127 and the film 144 is made so that the film 144 and the support 143 form an overhang 145 over the blanks 127, at least at the right-hand side edge 132 and the left-hand side edge 133 of the blanks 127 respectively.

After the application 620, the method 600 comprises a compression 630, at least of the overhangs 145 for moisture-repellent connection of the support 143 with the film 144. The compression 630 can, for example, comprise an ultrasound weld.

After the compression 630 a separation 640 of the combined system of support 143, inner regions 126 and film 144, takes place, for example by a cutting of the combined system, into individual transport means 120, which in particular can form a complete time measurement apparatus 100 in each case.

In particular, the separation 640 can take place in such a way that the inner regions 126 are freely accessible, at least at a lower edge 135 and, in particular, also at an upper edge 134 of the relevant transport means 120, at least in sections.

FIG. 7 shows a schematic illustration of a filter paper web 419 according to embodiments of the invention, for example made from polylactides, for volume production of teabags 420. On the filter paper web 419 is fastened a time measurement apparatus 100 for each teabag 420, wherein the rear surface of the transport means 120 of the time measurement apparatus 100, which can be coated, for example, with a thermoplastic material, is fastened to the filter paper web 419, at least by at least one lower fastening 418, for example an ultrasound weld, near the lower edge 135 of the transport means 120.

The rear face 131 of the transport means 120 of the time measurement apparatus 100 is fastened at least by at least one upper fastening 417, for example an ultrasound weld, near the upper edge 134 of the transport means 120 on the filter paper web 419.

The filter paper web 419 for production of the teabag 420 can be foldable about a longitudinal axis of the filter paper web 419 in a central folding region 415 of the filter paper web 419, wherein the transport means 120 of the time measurement apparatus 100 is arranged transversally to the longitudinal axis on the filter paper web 419 and fastened to the filter paper web by a central fastening 416, for example an ultrasound weld, between the lower edge 135 of the transport means 120 and the central folding region 415.

Although the invention has been illustrated and described in greater detail with reference to the preferred exemplary embodiment, the invention is not limited to the examples disclosed, and further variations can be inferred by a person skilled in the art, without departing from the scope of protection of the invention.

For the sake of clarity, it is to be understood that the use of “a” or “an” throughout this application does not exclude a plurality, and “comprising” does not exclude other steps or elements.

### LIST OF REFERENCE CHARACTERS

100 Time measurement apparatus

120 Transport means

121 Reservoir region

122 Detection region

123 Time region

124 Entry surface

126 Inner region

127 Blank

130 Front surface

131 Rear surface

132 Right-hand side edge

133 Left-hand side edge

134 Upper edge

135 Lower edge

140 Outer region

142 Section

143 Support

144 Film

145 Overhang

160 Resistance element

181 Dye

300 Marking

400 Time measurement system

410 Vessel

415 Folding region

416 Central fastening

417 Upper fastening

418 Lower fastening

419 Filter paper web

420 Teabag

421 Squeezing device

422 Holding device

600 Production method

601 Printing

610 Provision

620 Application

630 Compression

640 Separation

1. A time measurement apparatus with a transport device for a liquid, in the form of a strip, at least one sensor and at least one display connected at least communicatively with the sensor to display a contact duration of the time measurement apparatus with the liquid or an item of meta information derived from the contact duration, wherein the transport device comprises:
a) at least one reservoir region for taking liquid from an environment of the time measurement apparatus through an entry surface arranged at a lower edge of the transport device;
b) at least one detection region for detection of the liquid; and
c) at least one time region with a number of channels to transport the liquid in a defined transport duration from the reservoir region to the detection region, and wherein the sensor is designed for detection of the liquid in the detection region at an end of the time region remote from the reservoir region;
wherein
d) the transport device comprises a combined system with an inner region to transport the liquid and a moisture-repellent outer region;
e) wherein the outer region encloses the inner region at a front surface of the transport device, at a rear surface of the transport device opposite the front surface, at a right-hand side edge of the transport device and at a left-hand side edge opposite the right-hand side edge, in each case at least in a section adjoining a lower edge for immersion in the liquid; and
g) wherein the inner region is freely accessible, at least in sections, at the lower edge (135) and at an upper edge of the transport device opposite the lower edge.

2. The time measurement apparatus according to claim 1, wherein at least one resistance element for limiting a flow of liquid from the reservoir region into the time region, wherein the resistance element relative to at least one of the reservoir region and the time region,
a) comprises a reduced cross-sectional area for passage of the liquid;
b) comprises at least one of a material with a reduced conductivity for the liquid; and
c) has an increased material density.

3. The time measurement apparatus according to claim 1, wherein the inner region of the transport device
a) forms the sensor and the display;
b) has a translucency variable by contact with the liquid, opaque in a dry state and translucent in a wet state; and
c) includes at least one of absorbent material for the liquid, paper and a non-woven fabric.

4. The time measurement apparatus according to claim 1, wherein the outer region
a) comprises at least one of a moisture-repellent support, a cardboard coated with a thermoplastic synthetic material, for stiffening of the transport device; and
b) comprises at least a moisture-repellent film, transparent at least in sections, made from a thermoplastic synthetic material.

5. The time measurement apparatus according to claim 1, wherein at least one closure device for temporary closure of the entry surface against a penetration of moisture into the reservoir region, wherein the closure device is soluble in the liquid, only above a minimum temperature.

6. The time measurement apparatus according to claim 4, wherein
a) the rear surface of the transport device is formed by the support;
b) the front surface of the transport device is formed by the film; and
c) the inner region of the transport device is arranged between the support and the film;
d) wherein the support and the film in each case form an overhang over the inner region at the side edges of the transport device;
e) wherein at least the support and the film are connected to form a material bond with each other, at least at the side edges; and
f) wherein, the inner region and the support are connected with each other to form a material bond, at least in sections.

7. The time measurement apparatus according to claim 1,
wherein
a) the display comprises a marking, a colored marking;
b) whereby the marking is applied to the inner region on at least one of a side of the inner region facing the rear surface and to the outer region;
c) so that the marking is visible through the inner region from the front surface in a wet state of the inner region; and
d) wherein the marking is separated from the inner region, by a moisture-repellent material, by at least one of a moisture-repellent film and by a moisture-repellent coating of a support.

8. A filter paper web for volume production of pyramidal, teabags, wherein
a) a time measurement apparatus according to claim 1 is fastened on the filter paper web for each teabag; and
b) wherein the rear surface of the transport device of the time measurement apparatus is fastened to the filter paper web at least by at least one lower fastening near to the lower edge of the transport device.

9. The filter paper web according to claim 8, wherein
a) the rear surface of the transport device of the time measurement apparatus is fastened to the filter paper web by at least one upper fastening near the upper edge of the transport device and/or
b) the filter paper web for producing the teabag can be folded about a longitudinal axis of the filter paper web in a central folding region of the filter paper web, wherein the transport device of the time measurement apparatus is arranged traversely to the filter paper web and is fastened to the filter paper web by a central fastening between the lower edge of the transport device and the central folding region.

10. The time measurement system with a pyramidal teabag,
wherein a time measurement apparatus according to claim 1 is fastened to the teabag, wherein the rear surface of the transport device of the time measurement apparatus is fastened to the teabag, at least by a lower fastening near to the lower edge of the transport device.

11. The time measurement method with a time measurement apparatus according to claim 1, wherein
a) bringing the entry surface of the reservoir region of the transport medium of the time measurement apparatus into contact with a liquid at a contact start time;
b) transporting the liquid through the time region of the transport medium within a transport duration into the detection region of the transport medium,
c) detecting the liquid in the detection region at a detection time, which is correlated with the contact start timer, and
d) displaying the contact duration or an item of meta information derived from the contact time.

12. The time measurement method according to claim 11, wherein the detection comprises an optical, visual, detection of a change of the translucency of the inner region of the transport device of the time measurement apparatus caused by the liquid.

13. The time measurement according to claim 11, wherein the bringing into contact comprises an opening of a closing device for temporary closure of the entry surface against a penetration of moisture into the reservoir region by the liquid.

14. A production method for a time measurement apparatus according to claim 1,
wherein
a) provision of a blank for an inner region to transport the liquid;
b) application of one section each of a moisture-repellent outer region at least on a front surface and on a rear surface opposite the front surface of the blank;
c) wherein the sections form an overhang over the blank at a right-hand side edge of the blank and a left-hand side edge of the blank opposite the right-hand side edge in each case; and
d) compression at least of the overhangs for moisture-repellent connection of the sections with one another.

15. The production method according to claim 14, wherein the compression comprises
a) a weld, an ultrasound weld; and/or
b) a lamination, a hot lamination,
of the sections with one another and of at least one section with the blank.
